(19) United States
(12) Patent Application Publication
(10) Pub. No.: US 2024/0173450 A1
(43) Pub. Date: May 30, 2024

(54) Adjustable wax melting lamp

(71) Applicant: SHEN ZHEN LAMHO PHOTOELECTRICITY & TECHNOLOGY CO., LTD, Shenzhen (CN)

(72) Inventor: Yelin Gong, Fuzhou (CN)

(21) Appl. No.: 18/433,691

(22) Filed: Feb. 6, 2024

(51) Int. Cl. A61L 9/03

(52) U.S. Cl. CPC A61L 9/03; A61L 2209/11; A61L 2209/12; A61L 2209/15

(57) ABSTRACT

An adjustable wax melting lamp, including a placement base, an adjustment assembly, a lampshade, and a wax-melting lamp bead, wherein the adjustment assembly is provided on the placement base, the lampshade is provided on the adjustment assembly, and the wax-melting lamp bead is provided on the lampshade; the placement base is provided with a placement position for placing a candle; further comprising a main control board and a power supply, wherein the main control board is electrically connected to the wax-melting lamp bead and the power supply, and the main control board is electrically connected to a control module; and the placement base is provided with an ambient light source, and the placement base is made of a light-transmissive material.

### BACKGROUND OF THE INVENTION

### Technical Field

This disclosure relates to an adjustable wax melting lamp.

### Description of Related Art

The content disclosed herein merely provides background related to the invention and does not necessarily constitute the prior art.

With the progress of society and improvement of people's living standards, scented candles are used widely. Wax melting lamps used together with scented candles have both lighting and aromatherapy functions and are becoming increasingly popular. Traditional wax melting lamps usually include a lamp body support, a lamp and a scented candle. The lamp is installed on the lamp body support, and the scented candle contained in a container is positioned below the lamp. As the lamp continuously illuminate from top to bottom, it can melt the wax body and evaporates fragrance essential oil dispersed inside to form a fragrance.

However, existing wax melting lamps still have some shortcomings. Specifically, the positions of the lamps (wax melting lamps) of the existing wax melting lamps are usually fixed and cannot be adjusted. Therefore, consumers cannot adjust the wax melting speed and direction according to actual use needs and the actual situation of scented candles, which has brought inconvenience to some consumers. In addition, some existing wax melting lamps have limited functions and cannot well adapt to the needs of modern young people.

Therefore, a new technical solution is desired to solve the above problems.

### BRIEF SUMMARY OF THE INVENTION

In order to solve the above-mentioned defects and shortcomings of the prior art, the invention provides an adjustable wax melting lamp, where a lampshade is installed by providing an adjustable adjustment assembly on a placement base so that the position of the lampshade can be adjusted, making it more convenient to use the wax melting lamp. Moreover, an ambient light source is installed in the placement base, which further enriches the product's functions, meets the needs of more users, and greatly improves market competitiveness.

To achieve the above objective, the invention adopts the following technical solutions:

An adjustable wax melting lamp, including a placement base, an adjustment assembly, a lampshade, and a wax-melting lamp bead, wherein the adjustment assembly is provided on the placement base, the lampshade is provided on the adjustment assembly, and the wax-melting lamp bead is provided on the lampshade; the placement base is provided with a placement position for placing a candle;

further including a main control board and a power supply, wherein the main control board is electrically connected to the wax-melting lamp bead and the power supply, and the main control board is electrically connected to a control module; and the placement base is provided with an ambient light source, and the placement base is made of a light-transmissive material.

In some embodiments, the ambient light source includes a plurality of RGB lamp beads, and the plurality of RGB lamp beads are provided in the placement base at intervals along peripheral side walls of the placement base.

In some embodiments, the placement position is configured as a downwardly concave positioning groove.

In some embodiments, a balancing weight is provided in the placement base.

In some embodiments, the placement base is provided with a power supply port for powering electronic devices.

In some embodiments, the adjustable wax melting lamp further includes a wireless remote control, and the main control board has a wireless control module adapted to the wireless remote control.

In some embodiments, the adjustment assembly includes a first fixed rod, a first telescopic rod and a locking cap; the first fixed rod has an first through cavity extending up and down, the first telescopic rod is provided in the first through cavity in a manner of being telescopic up and down, a plurality of locking pawls are provided on an upper side of the first fixed rod, and the locking cap is provided corresponding to the locking pawls.

In some embodiments, the adjustment assembly includes at least two connecting rods and at least three steering balls, wherein the two connecting rods are connected by virtue of a steering ball, one of the connecting rods is connected to the placement base by virtue of a steering ball, and the other connecting rod is connected to the lampshade by virtue of a steering ball.

In some embodiments, the placement base is shaped like a rectangular parallelepiped, and the control module includes control buttons provided on a side wall of the placement base.

In some embodiments, the adjustment assembly includes a second fixed rod, a second telescopic rod and a locking screw; the second fixed rod has a second through cavity extending up and down, the second telescopic rod is provided in the second through cavity in a manner of being telescopic up and down, a side wall of the second fixed rod is provided with a locking through hole penetrating to the second through cavity, and the locking screw is provided corresponding to the locking through hole.

In some embodiments, the placement base is oval-shaped.

Compared with the prior art, the invention has obvious advantages and beneficial effects. Specifically, as can be seen from the above technical solutions, the invention main has its lampshade installed by providing the adjustable adjustment assembly on the placement base so that the position of the lampshade can be adjusted, making it more convenient to use the wax melting lamp. Moreover, the ambient light source is installed in the placement base, which further enriches the product's functions, meets the needs of more users, and greatly improves market competitiveness.

In addition, by providing the wireless control module, consumers can operate the lamp remotely, making it more convenient to use and more suitable for use in the room.

In order to illustrate the structural features and effects of the invention more clearly, the invention will be described in detail below with reference to the accompanying drawings and specific embodiments.

### BRIEF DESCRIPTION OF THE SEVERAL VIEWS OF THE DRAWINGS

FIG. 1 is a schematic perspective view of a first embodiment according to the invention;

FIG. 2 is a schematic cross-sectional view of the first embodiment according to the invention;

FIG. 3 is a partially enlarged view of FIG. 2;

FIG. 4 is a schematic perspective view of a second embodiment according to the invention; and

FIG. 5 is a schematic perspective view of a third embodiment according to the invention.

### REFERENCE NUMERALS

10. placement base; 101. control button; 102. charging port; 103. power supply port; 11. placement position; 12. main control board; 13. power supply; 14. ambient light source; 15. balancing weight; 16. wire; 161. controller; 20. adjustment assembly; 201. first fixed rod; 202. first telescopic rod; 203. locking cap; 204. connecting rod; 205. steering ball; 206. second fixed rod; 207. second telescopic rod; 208. locking screw; 30. lampshade; 40. wax-melting lamp bead.

### DETAILED DESCRIPTION OF THE INVENTION

The technical solutions in the embodiments of the invention will be clearly and completely described below with reference to the accompanying drawings. Obviously, the described embodiments are only preferred embodiments of the invention.

It should be noted that, when an element is described as “fixed on” another element, the element may be directly arranged on another element or there may be an intermediate element therebetween. When an element is considered as “connected” to another element, the element may be directly connected to another element or there may also be an intermediate element therebetween. The terms “vertical”, “horizontal”, “left”, “right” and similar expressions used herein are for illustrative purposes only and do not represent the only implementation manner.

Unless otherwise defined, all technical and scientific terms used herein have the same meaning as commonly understood by those skilled in the technical field to which the invention belongs. Terms used in the description of the invention herein are only for describing specific embodiments, not for limiting the invention. The term “and/or” used herein includes any and all combinations of one or more related listed items.

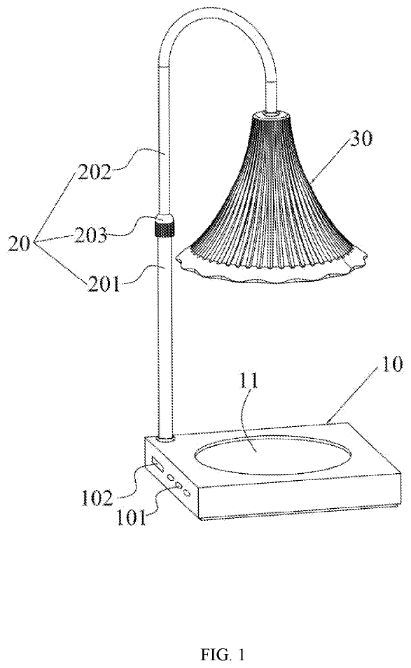

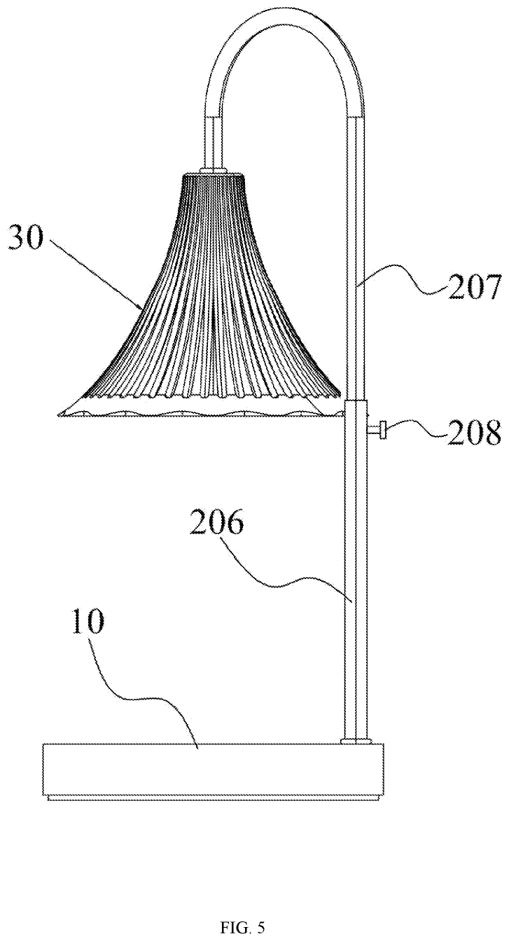

Referring FIG. 1 to FIG. 5, an adjustable wax melting lamp in the embodiments of the invention includes a placement base 10, an adjustment assembly 20, a lampshade 30, and a wax-melting lamp bead 40, wherein the adjustment assembly 20 is provided on the placement base 10, the lampshade 30 is provided on the adjustment assembly 20, and the wax-melting lamp bead 40 is provided on the lampshade 30; the placement base 10 is provided with a placement position 11 for placing a candle;

further includes a main control board 12 and a power supply 13, wherein the main control board 12 is electrically connected to the wax-melting lamp bead 40 and the power supply 13, and the main control board 12 is electrically connected to a control module; and the placement base 10 is provided with an ambient light source 14, and the placement base 10 is made of a light-transmissive material.

Typically, the ambient light source 14 includes a plurality of RGB lamp beads, and the plurality of RGB lamp beads are provided in the placement base 10 at intervals along peripheral side walls of the placement base 10. The placement position 11 is preferably configured as a downwardly concave positioning groove, thereby facilitating the positioning and placement of the candle.

Preferably, the placement base 10 is provided with a balancing weight 15. The balancing weight 15 may be an iron block, a stone block, or a cement block. Preferably, the lampshade 30 is flower-shaped, which not only blocks light from escaping, but also makes the overall product more beautiful, further improving the market competitiveness of the product. Preferably, the placement base 10 is provided with a power supply port 103 for powering electronic devices (mobile phones, tablet computers, etc.). The power supply port 103 may be configured as a USB port, a Type-C port, a Micro USB port, or the like.

Preferably, the adjustable wax melting lamp further includes a wireless remote control, and the main control board 12 has a wireless control module adapted to the wireless remote control. The main control board 12 may be correspondingly equipped with an infrared receiver to adapt to the wireless remote control, thereby realizing long-distance control and bringing convenience for consumers to use the lamp before going to bed. Further, the power supply 13 may be a battery installed in the placement base 10, and the main control board 12 is provided with a charging port 102 for charging a battery. Of course, the main control board 12 may also be provided with a wire 16 for connection with an external power supply 13, bringing convenience in use. The control module may include a controller 161 provided on the wire 16, making it more convenient for consumers to use the lamp.

In actual application, the placement base 10 may be oval-shaped or shaped like a rectangular parallelepiped.

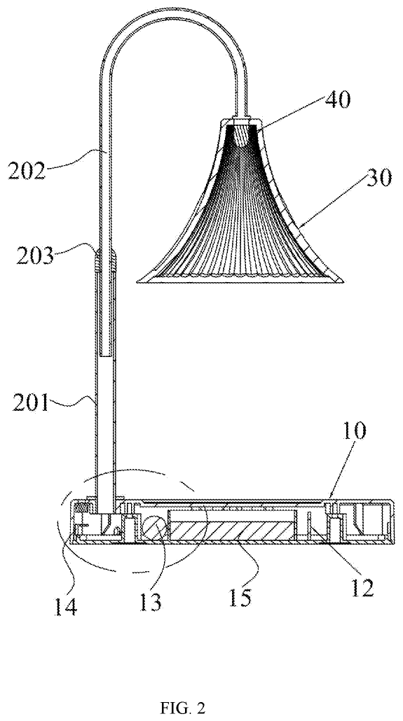

Referring to FIG. 1 and FIG. 2, as a preferred embodiment, the adjustment assembly 20 includes a first fixed rod 201, a first telescopic rod 202 and a locking cap 203; the first fixed rod 201 has an first through cavity extending up and down, the first telescopic rod 202 is provided in the first through cavity in a manner of being telescopic up and down, a plurality of locking pawls are provided on an upper side of the first fixed rod 201, and the locking cap 203 is provided corresponding to the locking pawls. In some embodiments, the placement base 10 is shaped like a rectangular parallelepiped, and the control module includes control buttons 101 provided on a side wall of the placement base 10.

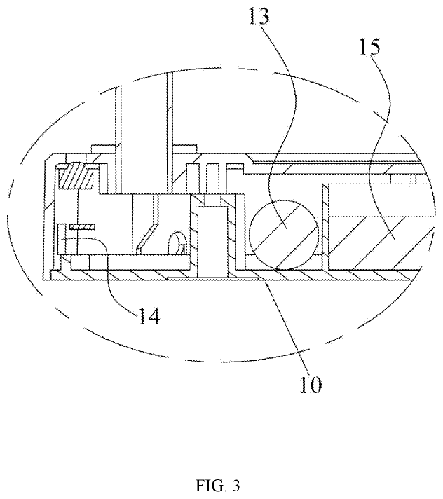

Referring to FIG. 3, as a preferred embodiment, the adjustment assembly 20 includes at least two connecting rods 204 and at least three steering balls 205, wherein the two connecting rods 204 are connected by virtue of a steering ball 205, one of the connecting rods 204 is connected to the placement base 10 by virtue of a steering ball 205, and the other connecting rod 204 is connected to the lampshade 30 by virtue of a steering ball 205. In this embodiment, the placement base 10 is oval-shaped.

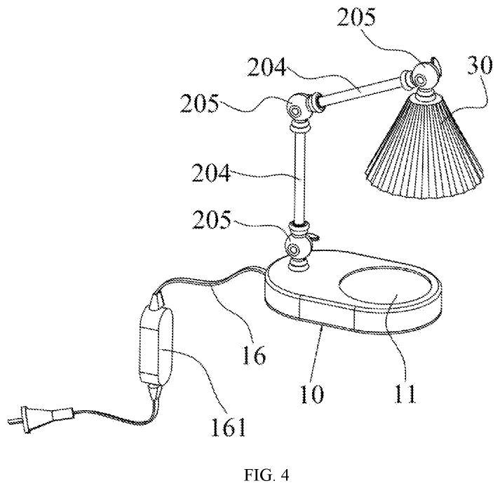

Referring to FIG. 4, as a preferred embodiment, the adjustment assembly 20 includes a second fixed rod 206, a second telescopic rod 207 and a locking screw 208; the second fixed rod 206 has a second through cavity extending up and down, the second telescopic rod 207 is provided in the second through cavity in a manner of being telescopic up and down, a side wall of the second fixed rod 206 is provided with a locking through hole penetrating to the second through cavity, and the locking screw 208 is provided corresponding to the locking through hole.

The design of the invention is characterized in that the lampshade is installed by providing the adjustable adjustment assembly on the placement base so that the position of the lampshade can be adjusted, making it more convenient to use the wax melting lamp. Moreover, the ambient light source is installed in the placement base, which further enriches the product's functions, meets the needs of more users, and greatly improves market competitiveness.

In addition, by providing the wireless control module, consumers can operate the lamp remotely, making it more convenient to use and more suitable for use in the room.

The above are only preferred embodiments of the invention and do not limit the technical scope of the invention in any way. Therefore, any minor amendments, equivalent changes and modifications made to the above embodiments according to the technical essence of the invention will still fall within the scope of the technical solution of the invention.

What is claimed is:

1. An adjustable wax melting lamp, comprising a placement base, an adjustment assembly, a lampshade, and a wax-melting lamp bead, wherein the adjustment assembly is provided on the placement base, the lampshade is provided on the adjustment assembly, and the wax-melting lamp bead is provided on the lampshade; the placement base is provided with a placement position for placing a candle;
further comprising a main control board and a power supply, wherein the main control board is electrically connected to the wax-melting lamp bead and the power supply, and the main control board is electrically connected to a control module; and the placement base is provided with an ambient light source, and the placement base is made of a light-transmissive material.

2. The adjustable wax melting lamp according to claim 1, wherein the ambient light source comprises a plurality of RGB lamp beads, and the plurality of RGB lamp beads are provided in the placement base at intervals along peripheral side walls of the placement base.

3. The adjustable wax melting lamp according to claim 2, wherein the placement position is configured as a downwardly concave positioning groove.

4. The adjustable wax melting lamp according to claim 3, wherein a balancing weight is provided in the placement base.

5. The adjustable wax melting lamp according to claim 4, wherein the placement base is provided with a power supply port for powering electronic devices.

6. The adjustable wax melting lamp according to claim 4, further comprising a wireless remote control, wherein the main control board has a wireless control module adapted to the wireless remote control.

7. The adjustable wax melting lamp according to claim 5, wherein the adjustment assembly comprises a first fixed rod, a first telescopic rod and a locking cap; the first fixed rod has an first through cavity extending up and down, the first telescopic rod is provided in the first through cavity in a manner of being telescopic up and down, a plurality of locking pawls are provided on an upper side of the first fixed rod, and the locking cap is provided corresponding to the locking pawls.

8. The adjustable wax melting lamp according to claim 5, wherein the adjustment assembly comprises at least two connecting rods and at least three steering balls, wherein the two connecting rods are connected by virtue of a steering ball, one of the connecting rods is connected to the placement base by virtue of a steering ball, and the other connecting rod is connected to the lampshade by virtue of a steering ball.

9. The adjustable wax melting lamp according to claim 8, wherein the placement base is shaped like a rectangular parallelepiped, and the control module comprises control buttons provided on a side wall of the placement base.

10. The adjustable wax melting lamp according to claim 5, wherein the adjustment assembly comprises a second fixed rod, a second telescopic rod and a locking screw; the second fixed rod has a second through cavity extending up and down, the second telescopic rod is provided in the second through cavity in a manner of being telescopic up and down, a side wall of the second fixed rod is provided with a locking through hole penetrating to the second through cavity, and the locking screw is provided corresponding to the locking through hole.

11. The adjustable wax melting lamp according to claim 10, wherein the placement base is oval-shaped.
